(19) United States
(12) Patent Application Publication
(10) Pub. No.: US 2026/0311828 A1
(43) Pub. Date: Oct. 8, 2026

(54) SYSTEMS AND METHODS FOR PATIENT PAIN IDENTIFICATION, TRACKING, AND VISUALIZATION

(71) Applicant: REGENTS OF THE UNIVERSITY OF MICHIGAN, Ann Arbor, MI (US)

(72) Inventors: Alexandre DaSilva, Ann Arbor, MI (US); Xiaosu Hu, Ann Arbor, MI (US); Fabio Thiers, Brooklyn, NY (US)

(21) Appl. No.: 19/636,008

(22) Filed: Apr. 1, 2026

Related U.S. Application Data
(60) Provisional application No. 63/782,457, filed on Apr. 2, 2025.

(51) Int. Cl. G16H 10/60; G16H 15/00; G16H 50/20

(52) U.S. Cl. CPC G16H 10/60; G16H 15/00; G16H 50/20

(57) ABSTRACT

Systems and methods for identifying, tracking, and visualizing pain within a patient are disclosed herein. A method may include obtaining data regarding a condition of a first user, the data received by providing, via a client device, natural language prompts and receiving, natural language responses; extracting, using a large language model, from the natural language responses, user medical information describing the condition of the user and conforming to a data structure of a medical record database; storing the user medical information in the medical record database; obtaining, via a second client device of a second user, further natural language responses describing the condition of the first user; via the one or more large language models, matching the further natural language responses to the user medical information; and providing an output indicating the user medical information to the second user.

### CROSS REFERENCE TO RELATED APPLICATIONS

This application claims priority to and the benefit of the filing date of provisional U.S. Patent Application No. 63/782,457 entitled “Systems and Methods for Patient Pain Identification, Tracking, and Visualization” filed on Apr. 2, 2025. The entire contents of the provisional application are hereby expressly incorporated herein by reference.

### STATEMENT OF GOVERNMENT INTEREST

This invention was made with government support under EB033664 awarded by the National Institutes of Health. The government has certain rights in this invention.

### TECHNICAL FIELD

The present disclosure generally relates to large language models (LLMs), and more specifically, to systems and methods for using an LLM to identify, track, and visualize aspects of a medical condition of a patient.

### BACKGROUND OF THE DISCLOSURE

To diagnose and monitor conditions of patients (e.g., conditions involving chronic pain), clinicians largely rely on patient self-assessment of conditions, for example via questionnaires or interviews conducted in advance of (and/or during) a clinical appointment. It can be difficult, however, for a patient to self-assess his or her condition, for example when assessing the condition involves describing the location, type, severity, and/or progression of pain and associated symptoms. Often, these self-assessments do not produce detailed or meaningful information that can be logged in an electronic system and used to inform a diagnoses and/or treatment plan. Furthermore, to the extent that a self-assessment is performed substantially in advance of the clinical appointment, the patient’s self-assessment (e.g., a description of pain) can be out of date by the time of the appointment. Thus, there is an unmet clinical need for a solution that effectively identifies and presents aspects of patient conditions such as chronic pain, for example to enable clinicians to make timely and accurate clinical decisions.

### SUMMARY

The present description identifies techniques involving a large language model (LLM) and/or other machine learning techniques to receive, analyze, and track real-time information from a patient regarding his or her condition (e.g., including chronic pain). Additionally, the present description identifies techniques for providing corresponding output regarding the patient’s condition to a clinician, e.g., via further interactions with the LLM (text or voice). Still additionally, the present disclosure identifies data visualization techniques for providing visual representations of the patient’s condition (including the progression thereof) to the patient and/or clinician(s).

In embodiments, a computer-implemented method performed via one or more processors may be provided. The computer-implemented method may include (1) obtaining real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts, (2) extracting, using one or more large language models, from the one or more natural language responses, user medical information describing the condition of the first user and conforming to a data structure of a medical record database, (3) storing the user medical information in the medical record database, (4) obtaining, via a second client computing device of a second user, a further one or more natural language responses describing the condition of the first user, (5) via the one or more large language models, matching the further one or more natural language responses to at least a portion of the user medical information, and (6) providing an output indicating the user medical information to the second client computing device of the second user.

In embodiments, a computer system is provided. The computer system may include one or more processors and one or more memories and may cause the one or more processors to (1) obtain real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts, (2) extract, using one or more large language models, from the one or more natural language responses, user medical information describing the condition of the first user and conforming the user medical information to a data structure of a medical record database, (3) store the user medical information in the medical record database, (4) obtain, via a second client computing device of a second user, a further one or more natural language responses describing the condition of the first user, (5) via the one or more large language models, match the further one or more natural language responses to at least a portion of the user medical information, and (6) provide an output indicating the user medical information to the second client computing device of the second user.

In embodiments, a non-transitory computer readable medium is provided. The non-transitory computer readable medium may store non-transitory computer executable instructions that, when executed by a processor include (1) obtaining real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts, (2) extracting, using one or more large language models, from the one or more natural language responses, user medical information describing the condition of the first user and conforming to a data structure of a medical record database, (3) storing the user medical information in the medical record database, (4) obtaining, via a second client computing device of a second user, a further one or more natural language responses describing the condition of the first user, (5) via the one or more large language models, matching the further one or more natural language responses to at least a portion of the user medical information, and (6) providing an output indicating the user medical information to the second client computing device of the second user.

### BRIEF DESCRIPTION OF THE FIGURES

The figures described below depict various aspects of the system and methods disclosed herein. It should be understood that each figure depicts an aspect of a particular aspect of the disclosed system and methods, and that each of the figures is intended to accord with a possible aspect thereof. Further, wherever possible, the following description refers to the reference numerals included in the following figures, in which features depicted in multiple figures are designated with consistent reference numerals.

FIG. 1 depicts a schematic diagram of an example computer system, in accordance with some embodiments.

FIG. 2 depicts an example client computing device displaying a patient’s live interaction with an active chat bot, in accordance with some embodiments.

FIG. 3A depicts an example flow diagram in association with using a machine learning model to extract patient medical information from natural language responses, in accordance with some embodiments.

FIG. 3B depicts another example flow diagram in association with training and operating a machine learning model, in accordance with some embodiments.

FIG. 4 depicts another example interface displaying a 3-D human body map mesh model representing the condition of the patient, in accordance with some embodiments.

FIG. 5 depicts still another example interface associated with tracking symptoms associated with a condition, in accordance with some embodiments.

FIG. 6A depicts an example output interface displaying patient medical information, in accordance with some embodiments.

FIG. 6B depicts another example output interface displaying a magnified version of patient medical information, in accordance with some embodiments.

FIG. 7A depicts still another example interface relating to live interaction with an active chat bot to retrieve patient medical information, in accordance with some embodiments.

FIG. 7B depicts another example interface displaying live interaction with an active chat bot to retrieve patient medical information, in accordance with some embodiments.

FIG. 8 depicts an example patient profile interface, in accordance with some embodiments.

FIG. 9 depicts another example output interface displaying changes in a level of pain and location of the pain of a patient over a course of time, in accordance with some embodiments.

FIG. 10 depicts a flow diagram of an example method, in accordance with some embodiments.

### DETAILED DESCRIPTION

Embodiments of the present description relate to computing systems and methods that, broadly speaking, use a large language models (LLM) to identify, track, and visualize aspects of a medical condition of a patient. According to certain aspects, an LLM may be configured to collect complex real-time data on a patient’s condition (e.g., chronic pain), for example via natural language communications with the patient via the patient’s personal electronic device. In embodiments, the LLM may analyze and convert natural language description of the patient’s condition into clinical data matching a data structure of a medical record database, to enable applications and individuals to more effectively process and utilize information describing the patient’s condition (e.g., to make timely and accurate clinical decisions). In still further aspects of the present description, one or more applications at a client device of a clinician may enable a clinician to interact with the LLM to obtain an up-to-date description of the patient’s condition, for example in the form of a clinical chart and/or a natural language description of the patient’s condition (e.g., in advance of and/or during a clinical appointment for the patient). In particular, in some embodiments, the clinician may access and navigate a three-dimensional (“3-D”) virtual model of the patient to examine various aspects of the patient’s condition (e.g., location, severity, and/or type of pain), and still further in some embodiments, may access and navigate a “four-dimensional” (or “4-D”) model comprising respective 3-D representations of a patient model over time to represent the progress of the condition of the patient.

Systems and methods described herein therefore offer a number of benefits. For example, the use of various machine learning techniques enables the systems and methods to accurately, more consistently, and dynamically identify and track a medical condition of a patient. This is particularly beneficial in situations in which conventional diagnoses consume substantial time and effort and for which conventional techniques for obtaining and analyzing patient information may be insufficient (e.g., where a diagnosis is based on subjective interpretation of data with high degrees of error). Additionally, it can be very difficult and somewhat unreliable for a patient to self-assess his or her condition. For example, when assessing the condition involves describing the location, type, severity, and/or progression of pain and associated symptoms, patients often do not produce detailed or meaningful information that can be logged in an electronic system and used to inform a diagnoses and/or treatment plan. Thus, the use of LLM techniques to obtain information regarding the patient’s condition (e.g., via natural language communications with the patient) may enable systems and methods herein to obtain more meaningful and complete information regarding the patient’s condition and/or the progression thereof. Moreover, visualization techniques described herein may improve graphical user interface technology by, for example, using the 3-D or 4-D model to visualize and navigate various aspects of the patient’s condition (e.g., zoom in or out, or rotate a particular body region), integrating in some embodiments further LLM functionalities to enable a clinician or patient to more effectively navigate the 3-D or 4-D model. These and other benefits will be understood further in the present description of these systems and methods.

Additionally, the use of the various machine learning techniques enables the systems and methods to accurately, more consistently, and dynamically determine proper diagnosis and treatment of patient diseases and conditions. Thus, the systems and methods herein may lead to the preparation of more accurate and effective treatment plans, improving the probability and speed of patient recovery from identified conditions. These benefits may decrease the strain on healthcare systems and reduce the costs incurred by patients and other individuals. It should be appreciated that additional benefits are envisioned.

### EXAMPLE COMPUTING SYSTEM

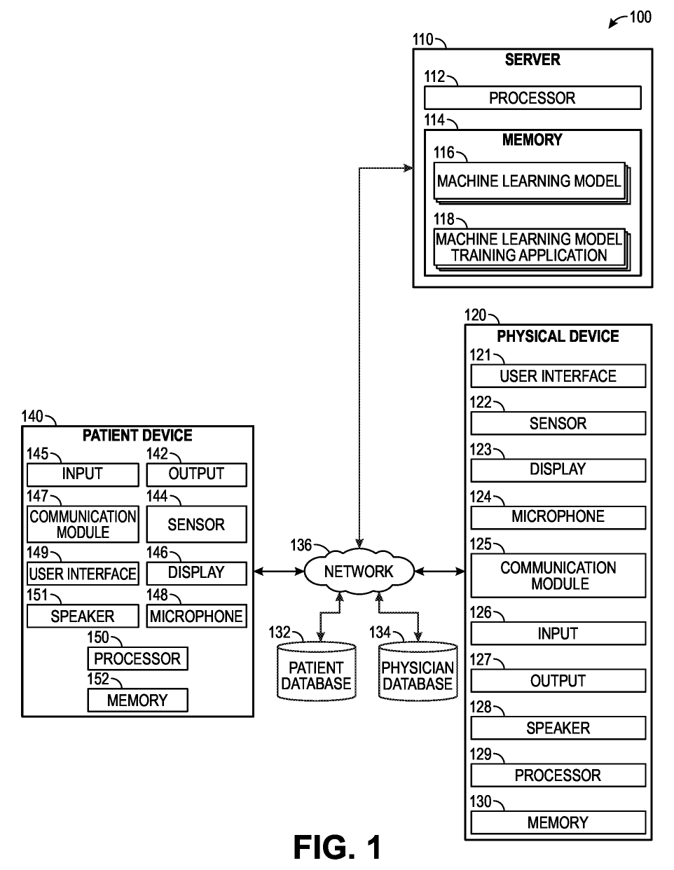

FIG. 1 depicts a schematic diagram of a computer system 100, in accordance with some embodiments. It should be appreciated that the system 100 is merely an example, and that alternative or additional components may be envisioned, in various embodiments. The system 100 illustrated in FIG. 1 may include both hardware and software components, as well as various data communications channels for communicating data between the various hardware and software components, as is described below.

The system 100, at a high level, may include a server 110 (i.e., one or more servers), an electronic computing device 120 associated with a physician (hereinafter “physician device”), and/or an electronic computing device 140 associated with a patient (“patient device”). Each of the physician device 120 and patient device 140 may include, for example, a smart phone, a smart watch or fitness tracker device, a tablet, a laptop, a virtual reality headset, a smart or augmented reality glasses device, another wearable device, etc.). The server 110, physician device 120, and/or patient device 140 may be configured to communicate with one another (and/or with other portions of the system 100) via a wired or wireless computer network 136, and/or via short range signals, such as Bluetooth signals. Although the term “physician device” is used herein, it should be appreciated that the physician device 120 may refer to an electronic computing device accessed via any suitable persons associated with identification, analysis, treatment, etc. of a condition of a patient (e.g., medical office personnel, the patient himself/herself, etc.). Likewise, although the term “patient device” is used herein, it should be appreciated that the patient device 140 may be accessed by one or more persons other than the patient, in some embodiments (e.g., a parent or other caregiver of the patient). Thus, where a user of the patient device 140 is referred to as a “patient,” it should be appreciated that in embodiments the patient may be substituted with another user who interacts with the patient device 140 (e.g., while acting on behalf of the patient as a patent or caregiver).

Although one server 110, one physician device 120, one patient device 140, and one network 136 are shown in FIG. 1, any number of such servers 110, physician devices 120, patient devices 140, and networks 136 may be included in various embodiments. For example, a patient or person associated therewith may perform actions described herein via a combination of multiple patient devices 140. Likewise, a physician and/or other medical personnel may perform actions described herein via a combination of multiple physician devices 120. To facilitate communications among the devices of FIG. 1, the server 110, physician device 120, and/or patient device 140 may each respectively comprise a wireless transceiver to receive and transmit wireless communications.

The server 110 of the system 100 may include a processor 112 (i.e., one or more processors), and/or a memory 114 (i.e., one or more memories). In some embodiments, the server 110 may comprise multiple, redundant, or replicated servers as part of a server farm. In still further aspects, the server 110 may be implemented as one or more cloud-based servers, such as a cloud-based computing platform. For example, such one or more servers may use one or more cloud-based platforms such as Microsoft Azure, Amazon Web Services (AWS), or the like. Each of such one or more servers may include a processor (i.e., one or more processors, e.g., CPUs) as well as a memory (i.e., one or more memories).

The memory 114 of the server 110 may store a machine learning model 116 (i.e., one or more machine learning models,), and/or a machine learning model training application 118 (i.e., one or more machine learning model training applications). The memory 114 of the server 110 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 114 may store an operating system (OS) (e.g., Microsoft Windows, Linux, UNIX, etc.) capable of facilitating the functionalities, apps, methods, or other software as discussed herein. The machine learning model 116 may include, for instance, one or more LLMs and/or other machine learning models trained to generate natural language queries and/or other communications, analyze one or more natural language responses and/or other responses to one or more prompts (e.g., natural language prompts), extract from the one or more natural language responses patient medical information describing the condition of the patient, conform the patient medical information to a data structure of a medical record database, and/or perform various other actions described herein.

Still additionally, or alternatively, the memory 114 may store natural language response data, medical records describing the condition of the patient, and/or other described in association with functionalities herein. Natural language response data may include, for instance, textual and/or audio responses to prompts by the patient and/or another person(s) associated therewith. In some embodiments, response data stored at the memory 114 may further include images responsive to one or more prompts (e.g., capturing a portion of a body of a patient in response to a prompt regarding a condition of the patient). The natural language and/or other response data may be stored in a patient database 132 (i.e., one or more databases), which may be included in or otherwise communicatively coupled to the server 110. Stored medical records describing the condition of the patient (and/or other information about the patient) may include text, image and/or video data from a patient describing a present and/or past condition of the patient, and may conform to a structure of a medical record database (e.g., defined by corresponding parameters, fields, etc., as the medical record database such as the patient database 132). In some embodiments, other user data (e.g., patient preferences, systems, profile information, etc.) may also be stored in the patient database 132.

The memory 114 may store instructions that, when executed by the processor 112, cause the processor 112 to receive data from the patient database 132, the patient device 140, and/or the physician device 120, e.g., in accordance with various functionalities described herein. The instructions stored on the memory 114, when executed by the processor 112, may cause the processor 112 to analyze data received from the patient database 132 and/or patient device 140 in order to make an identification or a prediction (e.g., an identification of a natural language response by a patient as the patient interacts with a chat feature (text or voice), an identification of information regarding a patient’s condition) (e.g., via natural language communications with the patient), identify past natural language responses by a patient describing the patient’s condition, conform patient medical information to a data structure of a medical record database, cause patient medical information to be stored the medical record database, indexed to body region(s) affected, and/or subsequently provide output to be displayed at the patient device 140 and/or at the physician device 120.

In some examples, the machine learning model 116 (e.g., one or more LLMs and/or another machine learning model(s)) may be executed on the server 110, while in other examples the machine learning model 116 may be executed on another computing system, separate from the server 110. For instance, the server 110 may send data to another computing system, where the machine learning model 116 is applied to the data, and the other computing system may send output of application of the machine learning model 116 to the server 110. In some examples, the machine learning model 116 may be trained by the machine learning model training application 118 executing on the server 110, while in other examples, a machine learning model 116 may be trained by another computing system, separate from the server 110.

The machine learning model training application 118 may train the machine learning model 116 using training data (e.g., indicating anonymized labeled historical data associated with medical conditions of patients identified based on natural language communications and/or other communications with said patients). The machine learning model 116, thus trained, may then be applied to new/current data that is separate from the training data in order to determine, e.g., predictions and/or identifications related to the new/current data (e.g., predictions or identifications of symptoms or conditions of a patient). The machine learning model 116 may be trained or re-trained by the machine learning model training application 118 using training data including text, audio, images, and/or videos of various natural language responses from the patient that are labeled with techniques used in each of the texts, audio, images, and/or videos.

Furthermore, a machine learning model 116 may be trained by the machine learning model training application 118 using training data including previously analyzed and tracked responses from the patient, that are labeled with known (e.g., formally diagnosed) patient conditions, symptoms, etc. For example, a machine learning model 116 may be trained by a machine learning model training application 118 using training data including audio responses of the patient’s medical condition and/or text responses from the patient regarding symptom(s) or pain level(s) associated with the various responses, and indications of level(s) of symptom(s) or pain and location(s) of pain. Moreover, a machine learning model 116 may be trained by a machine learning model training application 118 using training data including texts, audio, images, and/or videos of various further natural language responses from a physician or clinician that are labeled correct or preferred output responsive to the natural language responses. Once sufficiently trained using this training data, the machine learning model 116 may be applied to new natural language response(s) associated with a patient’s condition (e.g., audio captured by microphones 148 and 124, and/or text captured in real-time) and may predict or identify likely conditions of the patient or best treatments to assist the patient with their condition.

In various aspects, the machine learning model 116 may comprise, in an addition to one or more LLMs, other machine learning models such as neural networks, which may include deep learning neural networks, or combined learning modules or programs that learn in particular area(s) of interest. Aspects of the machine learning model 116 may still additional or alternatively include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other suitable machine learning algorithms and/or techniques. In some embodiments, the artificial intelligence and/or machine learning based algorithms used to train the machine learning model 116 may comprise a library or package executed on the server 110 (and/or on another computing device(s)). For example, such libraries may include the TENSORFLOW based library, the PYTORCH library, and/or the SCIKIT-LEARN Python library.

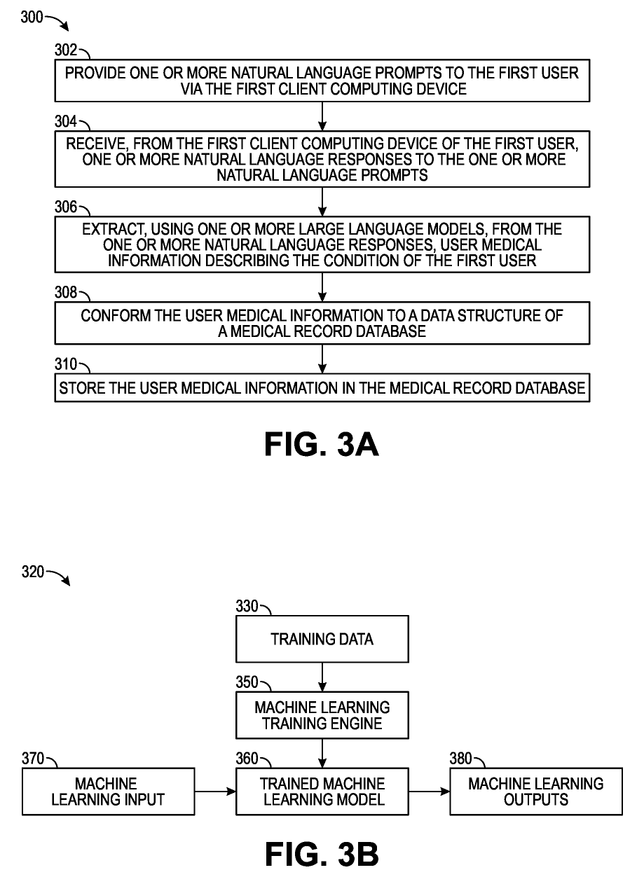

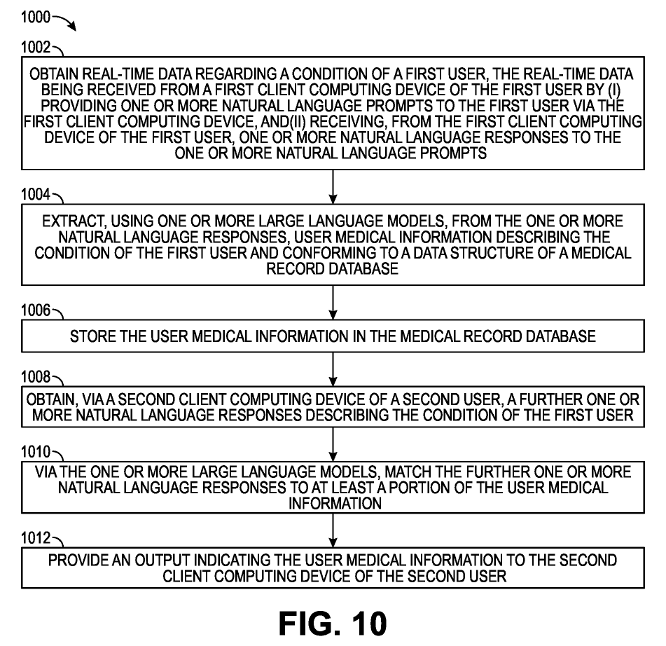

Still referring to FIG. 1, the memory 114 may also store additional machine readable instructions, including an application (e.g., one or more applications), a software component (e.g., one or more software components), and/or an application programming interface (API) (e.g., one or more application programming interface (APIs)), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For instance, in some examples, the computer-readable instructions stored on the memory 114 may include instructions for carrying out any of the steps of methods to be described with respect to FIGS. 3A and 10. It should be appreciated that one or more other applications may be envisioned and that are executed by the processor 112. It should be appreciated that, in some embodiments, some or all of described functions of the processor 112 may additionally or alternatively be implemented via a processor of another device, e.g., the physician device 120 and/or the patient device 140.

The patient device 140 may include a user interface 149, a sensor 144 (i.e., one or more sensors), a display 146, a microphone 148, a communication module 147, an input 145 (e.g., a mouse, keypad, touchscreen, etc.), an output 142, and/or a speaker 151 (i.e., one or more speakers). Moreover, the patient device 140 may include a processor 150 (i.e., one or more processors) as well as a computer memory 152 (i.e., one or more computer memories).

The patient device 140 may include or otherwise be operatively connected to the user interface 149. In some examples, the user interface 149 may include display 146, microphone 148, communication module 147, input 145, output 142, and/or speaker 151. In embodiments, any of the input 145, output 142, speaker 151, display 146, and/or microphone 148 may include one or more devices disposed within the patient device 140 and/or outside of the patient device 140 (e.g., wiredly or wirelessly connected to the patient device 140).

In some examples, the display 146 of the user interface 149 of the patient device 140 may include a touchscreen of a mobile computing device or of another client computing device. Generally speaking, the user interface 149, via the display 146, speaker 151, input 145, and/or microphone 148 collectively may provide a live chat feature for live interactions with a chat bot, and/or otherwise provide various information associated with a condition of a patient in accordance with techniques described herein. Received or provided information may include, for example, profile information of the patient, description of the condition or past conditions, and/or other information discussed in this detailed description. In some embodiments, the user interface 149, via the display 146, may provide a 3-D or 4-D human body map mesh model, with common data elements (e.g., multiple anatomically defined body cells), associated with the patient’s condition as to be described herein.

In some examples, the microphone 148 and/or speaker 151 (of the user interface 149 of the patient device 140 may capture, transmit, and/or play audio (e.g., one or more natural language prompts from a chat bot, one or more natural language responses from a patient, etc.). In some examples, the input 145 (i.e., one or more inputs) and/or output 142 (i.e., one or more outputs) may include, for example touchscreens, touchpads, keyboard, etc.

The user interface 149 of the patient device 140 may receive input from the patient and may provide audible or visible output to the patient (e.g., via the display 146, output 142, etc.). The user interface 149 may be configured to receive audio and visual inputs (e.g., via the input 145, sensor 144, microphone 148, etc.) and selections from the patient (e.g., using the input 145 and/or output 142), and/or to provide audible or visual feedback to the patient via the user interface 149, e.g., including instructions or guidance on inputting and/or retrieving information. For instance, the user interface 149 may be configured to receive audio inquiries (e.g., via microphone 148) regarding a patient’s medical information or a condition associated with a patient’s profile in order to retrieve accurate and real-time data regarding the patient’s condition, based on real-time data captured by the sensor 144. In some embodiments, the user interface 149 may receive input from the patient indicating a patient’s profile and a selection of a specific date and time associated with a condition of the patient. The selected patient profile may be associated with various parameters and/or specifications that the patient device 140 may use to allow a patient to provide a proper description or depiction of the patient’s symptoms and condition at the selected date and time.

In various examples, the user interface 149 may be configured to provide (e.g., via display 146 and/or speaker 151) visual or audio output associated with a selection made by the patient, such as a selected user profile, including a specific date, time, condition, symptom, history, etc. of the patient. This output may include an indication of the patient’s medical information, such as the current medical status report, which may range from a basic overview of the patient to specific aspects of a patient’s condition or symptoms, e.g., at a particular date, time, location of the body, etc. Furthermore, the user interface 149 may provide additional alerts, notifications, communications, etc., as discussed elsewhere herein.

In various examples, the user interface 149 may generate and display a 3-D or 4-D human body map mesh model to receive and/or display real-time information from the patient regarding the patient’s condition. For example, in some cases, the user interface 149 may visually display a 3-D or 4-D human body map mesh model of the patient’s condition as captured in real-time by the sensor 144, microphone 148, and/or input 145 of the patient device 140 to illustrate an identified or predicted characterization of a patient’s condition. In some examples, the user interface 149 may provide a highlighted or otherwise manipulated portion of the 3-D or 4-D human body map mesh model of the patient to illustrate that a medical condition (e.g., chronic pain) involves the highlighted or otherwise manipulated portion of the patient’s body (e.g., as detected via the machine learning model 116 based on natural language inputs).

Generally speaking, using the described 3-D model, a user may utilize a device (e.g., patient device 140) to visually display and explore a three-dimensional representation (“map mesh model”) of a patient’s body, the three-dimensional representation including visual elements representing particular conditions in particular portions of the patient’s body. A map mesh model may be defined by a suitable number of common data elements (CDEs) corresponding to respective body-cell coordinates, precisely defined based on anatomical and/or neurological regions of a patient’s body (e.g., dermatomes), where these regions are common to various patients such that the condition of any patient may be represented via manipulation of one or more suitable data elements in the map mesh model. In some example embodiments, the map mesh model includes over 2,300 data elements representing respective body-cell coordinates across various anatomical and/or neurological regions of the body of a patient. Moreover, in embodiments herein, one or more user interfaces may provide multiple time-based representations of the described 3-D model, effectively producing a 4-D model that represents the progression or other change in one or more conditions over time. Utilizing such one or more user interfaces, a user may navigate between respective 3-D representations for example to view the spread, intensification, reduction, and/or other change in characterization of one or more symptoms associated with a condition (e.g., intensification of knee pain based on varying 3-D “slices” of the 4-D model over time). Properties of respective data elements in the 3-D or 4-D model may be populated and displayed based upon reference to structured medical information of the user (e.g., from one or more databases), and/or based on natural language input from a patient, clinician, and/or other user upon analysis via the LLM techniques herein (which may, for example, convert said natural language input to corresponding manipulations of one or more data elements representing a portion of the patient’s body that is of particular concern).

In various embodiments, the 3-D or 4-D human body map mesh model may be configured to ensure consistency of cell-level mappings across different avatar types. For example, cell placement within the map mesh model may be defined relative to anatomical landmarks that are common across different body types, such that the mesh is landmark-anchored and corresponding cells represent the same anatomical regions regardless of the avatar type being displayed. In this manner, the common data elements (CDEs) may provide consistent mappings across male and female body models, adult and pediatric body models, and/or other variations in body type or morphology. This landmark-anchored approach may enable data captured from one avatar type to be compared with or mapped to data from another avatar type, as corresponding cells in each avatar represent equivalent anatomical regions. In some cases, this consistent mapping framework may facilitate reliable aggregation and cohort-level analysis of cell-based data collected from diverse patient populations, as medical information from patients of different body types may be aggregated and compared using the standardized anatomical coordinate system. Moreover, the landmark-anchored mesh architecture may support extensibility to additional avatar types developed in the future, enabling consistent data capture and comparison as new body model representations are introduced.

To provide a practical example, when a patient or clinician identifies and/or describes a left portion of the patient’s face as part of the patient’s medical condition, or location of pain in the case of chronic pain, the user interface 149 of the patient device 140 may produce and/or access a 3-D or 4-D human body map mesh model including highlighted and/or otherwise manipulated data elements corresponding to the left portion of the patient’s face. As another example, the patient or clinician may describe pain in the patient’s knee, and the user interface 149 of the patient device 140 may apply a highlighted section over corresponding data elements representing a knee of the patient. In still other various examples, the user interface 149 may generate a 3-D or 4-D human body map mesh model to represent symptoms in the patient’s mouth, tooth, face, arm, back, leg, chest, neck, foot, etc., via manipulation of suitable corresponding data elements. A user may select a particular portion of the patient’s body as displayed in 3-D or 4-D model to explore in more depth the condition of a particular portion of the patient’s body, and/or changes in condition, including the statistical changes specific to the region in focus, instead of the full body.

In some examples, the 3-D or 4-D human body map mesh model may include data elements corresponding to intraoral anatomy of the patient, enabling representation and tracking of conditions affecting the mouth, teeth, gums, tongue, palate, and other oral structures. For example, the map mesh model may include CDEs corresponding to individual teeth, quadrants of the mouth, gingival regions, and other intraoral anatomical landmarks. In some examples, a user may navigate the 3-D or 4-D human body map mesh model to zoom in on an intraoral region, enabling detailed examination of dental and oral conditions such as tooth pain, gum inflammation, temporomandibular joint (TMJ) disorders, oral lesions, and other orofacial symptoms. The user interface may receive natural language input from a patient or clinician describing intraoral symptoms (e.g., "pain in my upper left molar" or "sensitivity in my front teeth"), and the LLM may process such input to identify and manipulate corresponding data elements representing the described intraoral region.

In further examples, the intraoral mapping capabilities of the 3-D or 4-D human body map mesh model may support both medical and dental applications. For example, a dentist or oral surgeon may utilize the techniques herein to track the progression of periodontal disease across multiple teeth and gum regions over time, while a physician may utilize the same model to track orofacial pain symptoms associated with conditions such as trigeminal neuralgia or referred pain from other body regions. The user interface may provide specialized visualization tools for intraoral regions, including the ability to display individual tooth surfaces, cross-sectional views of dental structures, and color-coded representations of symptom intensity across different regions of the oral cavity. In some examples, the system may correlate intraoral symptoms with symptoms in other body regions represented by the map mesh model, enabling comprehensive tracking of conditions that manifest across multiple anatomical areas.

In various examples, the techniques described herein may be applied to aggregate and analyze medical information across multiple patients for cohort or population-level analysis. Because the 3-D or 4-D human body map mesh model utilizes CDEs that are standardized across patients, medical information from multiple patients may be aggregated and compared using a consistent anatomical framework. For example, a clinician or researcher may utilize the LLM to request aggregated information regarding a particular condition or symptom across a defined patient population (e.g., "show me the distribution of pain locations for patients with fibromyalgia" or "compare symptom progression across patients in a clinical trial"). In response, the system may process the request to identify and aggregate corresponding structured patient medical information from the medical record database across multiple patient records, and generate output providing population-level visualizations, statistics, and/or natural language summaries.

In further examples, the 3-D or 4-D human body map mesh model may be utilized to display aggregated or averaged representations of conditions across a patient cohort. For instance, the system may generate a composite body map mesh model illustrating the frequency, intensity, and/or distribution of symptoms across a population of patients, enabling identification of common patterns or trends. In some examples, the user interface may provide tools for defining patient cohorts based on various criteria (e.g., diagnosis, demographics, treatment regimen, etc.) and for comparing aggregated representations across different cohorts. These population-level analysis capabilities may facilitate multidisciplinary medical care, clinical research, epidemiological studies, and medical education by enabling comprehensive analysis and visualization of health conditions across individuals or populations.

The sensor 144 (i.e., one or more sensors) of the patient device 140, may be configured to capture real-time data associated with the patient’s condition (e.g., via the user interface 149 of the patient device 140). The sensor 144 may include, for instance, a camera and/or a depth sensor configured to capture data associated with the patient. Moreover, in some examples, the sensor 144 may include one or more sensing elements configured to capture biometric data associated with the patient, such as facial recognition data, fingerprint recognition data, iris recognition data, etc., e.g., for purposes of identification and/or for evaluation of a condition of the patient.

Generally speaking, the memory 152 may store instructions that, when executed by the processor 150, cause the processor 150 to receive an indication of a patient’s medical condition from a patient, and/or to generate and provide a 3-D or 4-D human body map mesh model (e.g., using techniques described herein). In embodiments, the memory 152 may store instructions that, when executed by the processor 150, cause the processor 150 to analyze audio, video, and/or text data captured by the sensor 144, input 145, microphone 148, etc., associated with the patient’s medical condition. For instance, the memory 152 may store instructions that, when executed by the processor 150, cause the processor 150 to capture image data (e.g., via the sensor 144) and analyze the image data to identify medical conditions associated with the patient.

In some embodiments, the memory 152 may store instructions that, when executed by the processor 150, cause the processor 150 to analyze audio, video, and/or text data captured by the sensor 144, input 145, microphone 148, etc., or send the audio, video, and/or text data captured by the sensor 144, input 145, microphone 148, etc. to the server 110, to be analyzed by a machine learning model 116 (e.g., using any of the machine learning techniques described herein). In some examples, the identified or predicted patient medical condition may be determined based on the patient’s previously reported conditions (e.g., as reported using any of the user interfaces described herein). Such identifications or predictions may for example pertain to whether or to what extent the patient is experiencing a medical condition or particular symptoms thereof, types or symptoms (e.g., location, level, and/or type of pain), etc.

Still additionally or alternatively, in embodiments, the instructions stored on the memory 152 may cause the processor 150 to analyze real-time natural language responses (e.g., as captured via any of the various components of the patient device 140 as described herein), for example in order to generate, display, navigate, and/or manipulate the 3-D or 4-D map mesh model according to the techniques herein. In some embodiments, these functionalities may integrate LLM functionalities to enable a physician or the patient to more effectively navigate or manipulate the 3-D or 4-D map mesh model (e.g., by providing text or voice commands to zoom in or out of, navigating between different portions of, or otherwise manipulating the 3-D or 4-D map mesh model). Still yet additionally or alternatively, in embodiments, the instructions stored on the memory 152 may cause a patient device 140 to provide audible or visual feedback or responses to the patient’s natural language responses, including instructions on inputting natural language responses, retrieving patient information, navigating or manipulating the 3-D or 4-D map mesh model, etc.

In examples, the processor 150 may execute instructions from the memory 152 to determine whether natural language prompts and/or other input provided by the patient and/or another user(s) is suitable to respond to one or more prompts. Upon the processor 150 determining that a user has sufficiently provided data (e.g., regarding the patient’s condition), the processor 150 may accordingly automatically proceed to subsequent steps described herein. In some examples, the instructions stored on the memory 152 may cause the processor 150 to extract, using the machine learning model 116, patient medical information describing the condition of the patient and conform the data to a data structure of a medical record database. In some examples, the instructions stored on the memory 152 may cause the processor 150 to store the patient medical information in the patient database 132.

Furthermore, in various examples, the instructions stored on the memory 152 may cause the processor 150 to perform any or all of the steps of methods to be described below with respect to FIGS. 3A and 10.

Referring still to FIG. 1, the physician device 120 may include a user interface 121, a sensor 122 (i.e., one or more sensors), a display 123, a microphone 124, a communication module 125, an input 126, an output 127, and/or a speaker 128 (i.e., one or more speakers). Moreover, the physician device 120 may include a processor 129 (i.e., one or more processors) as well as a computer memory 130 (i.e., one or more computer memories).

The physician device 120 may include or otherwise be operatively connected to the user interface 121. In some examples, the user interface 121 may include display 123, microphone 124, communication module 125, input 126, output 127, and/or speaker 128. In various embodiments, the above-described components of the physician device 120 may include components disposed within the physician device 120, and/or components otherwise operatively coupled to the physician device 120 (e.g., a wiredly or wirelessly connected mouse, keyboard, display, speaker, microphone, etc.).

In some examples, the display 123 of the user interface 121 of the physician device 120 may include a touchscreen of a mobile computing device or of another client computing device. Generally speaking, the user interface 121, via the display 123, may provide a live chat feature for live interactions with a chat bot, e.g., to request information from the physician and/or provide access to patient information. The requested and/or provided information may include, for example, the patient’s profile, description of the condition or past conditions, and/or other information discussed in this detailed description. In some embodiments, the user interface 121, via the display 123, may provide the physician with access to a 3-D or 4-D human body map mesh model associated with the patient’s condition.

In some examples, the microphone 124 (i.e., one or more microphones) and/or speaker 128 (i.e., one or more speakers) of the user interface 121 of the physician device 120 may capture, transmit, and/or play audio (e.g., one or more natural language prompts from a chat bot, one or more natural language responses from a physician, etc.). In some examples, the input 126 (i.e., one or more inputs) and/or output 127 (i.e., one or more outputs) may include, for example touchscreens, touchpads, keyboard, etc.

The user interface 121 of the physician device 120 may receive input from the physician and may provide audible and/or visual output to the physician (e.g., via the display 123, output 127, etc.). The user interface 121 may be configured to receive audio and visual inputs and from physician (e.g., using the input 126 and/or output 27), and/or to provide audible or visual feedback to the physician via the user interface 121, including for example instructions or guidance on inputting information associated with retrieving patient medical information. In examples , the user interface 121 may be configured to receive audio regarding a patient’s medical information or a condition associated with a patient’s profile in order to retrieve accurate and real-time data regarding a patient’s condition, based on real-time data captured by the sensor 122. In examples, relevant information retrieved by the physician device 120 includes data received from and/or displayed at a patient device 140, and/or information previously stored in a patient database 132.

In some embodiments, the user interface 121 may receive input from the physician indicating retrieval of a patient’s profile and a selection of a specific date and time associated with a condition of the patient. Relevant patient information may be retrieved, for example, from the patient database 132 and/or from a physician database 134. A selected patient profile may be associated with various parameters and/or specifications that the physician device 120 may use to allow the physician to display relevant information regarding the patient (e.g., a 3-D or 4-D human body map mesh model) and/or provide a proper diagnosis and treatment to the patient. Additionally, the user interface 121 may receive a text request or response, an image, or a video from the physician (e.g., via input 126, sensor 122, microphone 124, etc.).

Moreover, in some examples, the user interface 121 may be configured to provide visual or audio output associated with a selected patient profile, including a specific date, time, condition, symptom, history, etc. of the patient. This visual or audio output may include an indication of the patient’s medical information. The patient’s medical information may range, for example, from a basic overview of the patient’s medical information (e.g., a medical chart) to specific patient conditions, symptoms, dates/times or locations thereof, etc. Furthermore, in various examples, the user interface 121 may provide additional alerts, notifications, communications, etc., as discussed elsewhere herein.

In some examples, the user interface 121 may generate and/or provide, via the display 123, a 3-D or 4-D human body map mesh model representing a patient’s condition (e.g., in a manner analogous to that described with respect to the patient device 140). For example, in some cases, the user interface 121 may visually display the 3-D or 4-D human body map mesh model of the patient’s condition, based upon real-time information captured by the patient device 140 and/or physician device 120, and/or based upon other stored information (e.g., from the patient database 132 and/or physician database 134). In some examples, the user interface 121 may highlight or otherwise manipulate data elements representing a particular portion of the body of the patient as represented by the 3-D or 4-D human body map mesh model, to illustrate that an identified or predicted medical condition (e.g., chronic pain) involves the particular portion of the body. For example, referring back to the examples provided with respect to the patient device 140, the displayed 3-D or 4-D human body map mesh model may highlight or otherwise manipulate data elements corresponding to a patient’s face, knee, arm, foot, ear, etc., responsive to identification of one or more conditions and/or symptoms involving said portion(s) of the patient’s body.

The sensor 122 (i.e., one or more sensors) may be configured to capture real-time data associated with the physician’s request for patient medical information. The sensor 122 may include, for instance, a camera and/or a depth sensor configured to capture data associated with the physician. Moreover, in some examples, the sensor 122 may include one or more sensing elements configured to capture biometric data associated with the physician, such as facial recognition data, fingerprint recognition data, iris recognition data, etc., e.g., to identify patient conditions/symptoms and/or for security purposes.

Generally speaking, the memory 130 (i.e., one or more memories) may store instructions that, when executed by the processor 129, cause the processor 129 to perform various actions attributed to the physician device 120 herein. These actions may include, for example, receiving a request (e.g., a database request or natural language request) to retrieve a patient’s medical information and/or other description of a patient condition, and/or to generate and provide 3-D or 4-D human body map mesh model via the display 123. Additionally or alternatively, the memory 130 may store instructions to cause the processor 129 to analyze audio, video, and/or text data associated with the patient’s medical condition (e.g., as captured via techniques described herein, for example during an in-person visit to a physician and/or using the patient device 140).

In examples, the memory 130 may store instructions to cause the processor 129 to analyze audio, video, and/or text data captured by the physician device 120 and/or patient device 140, to be analyzed by a machine learning model 116, for example in order to identify or predict aspects of a patient’s medical condition via the LLM techniques described herein. In some examples, the identified or predicted patient medical condition may be determined based on the patient’s previously captured and documented conditions (as captured via various techniques of this disclosure), and/or may be determined based on the patient’s natural language responses (e.g., whether or to what extent the patient is experiencing a medical condition, whether or to what extend the patient is experiencing pain, whether or to what extent the patient is experiencing symptoms of a medical condition, whether or to what extent the patient has experienced a certain level of pain in the past and when, whether or to what extent the patient is able to fully describe the medical condition they are experiencing in real-time, etc.).

Furthermore, the instructions stored on the memory 130 may cause the processor 129 to analyze real-time natural language responses captured by one or more components of the physician device 120 and/or patient device 140 in order to generate, display, and/or manipulate (e.g., via natural language and LLM interactions) the 3-D or 4-D human body mesh map model representing a patient’s condition and/or progression thereof, for example via color differentiation, animation, highlighting, zooming in our out, etc.. In some examples, the instructions stored on the memory 130 may cause the processor 129 to analyze real-time natural language responses captured by one or more components of the physician device 120 and/or patient device 140, in order to generate a visual representation of the patient’s condition.

Additionally, the instructions stored on the memory 130 may cause a physician device 120 to provide audible or visual feedback or responses to the physician’s natural language responses, including for example instructions or guidance on inputting natural language responses. In examples, the user interface 121 may be configured to receive audio inquiries regarding a patient’s medical information or a condition associated with a patient’s profile in order to retrieve accurate and real-time data regarding the patient’s condition, based on real-time data captured by the sensor 122 and/or other components of the physician device 120 and/or patient device 140.

In examples, instructions from the memory 130 may cause the processor 129 determining determine whether natural language prompts and/or other input provided by the physician and/or another user(s) is suitable to respond to one or more prompts. Upon the processor 150 determining that a user has sufficiently provided data (e.g., regarding the patient’s condition, a request for access of a particular portion thereof, etc.), the processor 129 may accordingly automatically proceed to subsequent actions of the physician device 120 (e.g., to retrieve particular information, to display a particular portion of a 3-D or 4-D human body map mesh model, etc.). Still additionally or alternatively, in some examples, the instructions stored on the memory 130 may cause the processor 129 to extract, using a machine learning model 116, patient medical information describing the condition of the patient and conform the data to a data structure of a medical record database. In some examples, the instructions stored on the memory 130 may cause the processor 150 to store the patient medical information in the patient database 132.

Still yet additionally or alternatively, in some examples, the instructions stored on the memory 130 may cause the processor 129 to perform any or all of the steps methods to be described with respect to FIGS. 3A and 10.

### EXAMPLE CLIENT COMPUTING DEVICE INTERFACE

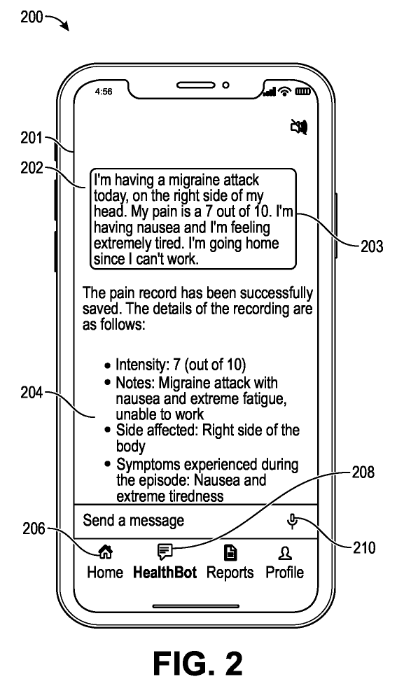

FIG. 2 depicts a client computing device 200 (e.g., patient device 140 from FIG. 1) displaying a patient’s live interaction with an active chat bot (referred to herein as “HealthBot”). More particularly, the client computing device 200 (hereinafter “patient device”) may include a display screen 201 (e.g., touchscreen) displaying a graphical user interface (GUI) 202. The GUI 202 includes a natural language input 203 (i.e., one or more natural language responses) from a patient or other user submitted via the techniques described herein, responsive to one or more prompts (not shown in FIG. 2). An LLM as described herein may process the natural language input 203 (e.g., locally at the patient device 200, and/or at one or more servers, for example server 110 of FIG. 1) to extract medical information of the patient from the natural language input 203. Extracted medical information may, for example, conform to a structure of a medical record database as described herein, e.g., including values in particular structured fields such as pain location, pain intensity, pain sensation type, pain progression, and/or other symptoms (e.g., nausea, migraine, anxiety, fatigue, lightheadedness, bleeding, depression, etc.) or signs (e.g., body lesion). Extracted medical information may include various information associated with pain conditions as described herein, e.g., symptoms of pain itself, and/or signs/symptoms indicative of or caused by the pain condition, e.g., nausea, level of stress, alcohol consumption, sleep quality, etc. In some embodiments, extracted medical information may include (and/or be derived from) information captured from the client computing device 200 (e.g., including sensor data or data derived from said sensor data). Responsive to receiving and processing the natural language input 203, the GUI 202 may display an output 204 indicating output of the LLM processing, e.g., identifying the particular structured fields extracted from the natural language input 203, with the output being generated by the LLM to answer the natural language input 203 in a casual, conversational manner.

In the example GUI 202 of FIG. 2, a navigation bar 206 is located at the bottom portion of the display screen 201 and may include one or more icons or buttons for navigating between screens, apps, and/or other features displayed on the patient device 200, etc. In examples, one of the apps and/or icons may include an icon 208 for navigation to the HealthBot functionality described herein.

In example implementations, the patient may use a microphone icon 210 to provide the natural language audio input describing the patient’s medical condition. The patient device 200 may then receive and process the received audio input and display the natural language input 203 via speech-to-text functionality.

In the example GUI 202 of FIG. 2, the natural language input 203 (and other patient input, where provided) is displayed on the right-hand side of the display screen 201, with responses via the HealthBot displayed on the left-hand side of the display screen 201. It should be appreciated, though, that in the GUI 202 and/or other GUIs described herein, various other arrangements are possible without fundamentally deviating from the techniques of this disclosure.

### EXAMPLE FLOW DIAGRAMS

FIG. 3A depicts a flow diagram of an example method 300 associated with using a machine learning model to extract patient medical information from natural language responses, in accordance with some embodiments. The method 300 may be performed, for example, via the patient device 140 of FIG. 1, patient device 200 of FIG. 2, and/or other patient device(s) of this disclosure. Performance of the method 300 via a patient device may include interaction of the patient device with an LLM which may be disposed locally at the patient device and/or at one or more servers (e.g., server 110 of FIG. 1).

The method 300 may include, at block 302, providing one or more natural language prompts to the first user via the first client computing device. At block 304, the method 300 may also include receiving from the first client computing device of the first user, one or more natural language responses to the one or more natural language prompts. At block 306, the method 300 may include extracting, using one or more large language models, from the one or more natural language responses, user medical information describing the condition of the first user. The method 300 may also include, at block 308, conforming the user medical information to a data structure of a medical record database. At block 310, the method 300 may include storing the user medical information in the medical record database.

FIG. 3B depicts another example flow diagram 320 associated with training and operating a machine learning model, such as an LLM described herein (i.e., one or more machine learning models, e.g., machine learning model 116 of FIG. 1), in accordance with some embodiments. Actions of the flow diagram 320 may be performed by the system 100, and more particularly by the server 110, in embodiments.

As depicted in FIG. 3B, training data 330 is provided to a machine learning training engine 350 (e.g., machine learning model training application 118 of FIG. 1). The machine learning training engine 350 may include one or more hardware and/or software components to obtain, create, (re)train, fine-tune, and/or store one or more machine learning models, thereby producing a trained machine learning model 360 based on the training data 330. A server, such as server 110, may obtain and/or have available one or more types of training data 330 (e.g., training data stored in the patient database 132 and/or the physician database 134). During training of the machine learning model 360 by the machine learning training engine 350, the machine learning model 360 may be configured to process the training data 330 to learn associations and relationships in the training data 330. More particularly, in examples, the training data 330 may include labeled data comprising natural language inputs from past patients and/or clinicians, with such responses being labeled with corresponding output indicative of correct patient medical information and/or desirable natural language structures for presenting such patient medical information to a user. Thus, by training based on the training data 330, the trained machine learning model 360 may identify relationships between the natural language inputs and appropriate outputs to thereby enable the trained machine learning model 360 to identify appropriate novel outputs 380 based on novel inputs 370 (e.g., to produce the various HealthBot interactions described herein).

In some embodiments, the training data 330 is updated as needed, e.g., to include new data. Such data may be stored as updated training data 330, based upon which the machine learning training engine 350 may re-train or tune the trained machine learning model 360, thereby improving the efficacy of the trained machine learning model 360 over time (e.g., make more accurate or precise identifications or predictions of patient conditions or symptoms, to more effectively communicate identifications or predictions to users via natural language outputs, and/or to more accurately respond to user requests to manipulate views of 3-D or 4-D human body map mesh models described herein).

In some embodiments, customized instantiations of the trained machine learning model 360 may be provided for respective users of HealthBot features described herein. In these embodiments, training data 330 for a respective instantiation of the trained machine learning model 360 may include labeled training data obtained over a course of use of the features herein by a particular user, e.g., where a user’s interactions are indicative of the user’s medical conditions, desired output, application usage preferences, and/or other aspects particular to the user. By training or re-training the trained machine learning model 360 based on this user-specific data, the techniques herein may learn and adapt to user preferences to improve subsequent usage of the LLM and/or other features herein.

In at least some aspects, the same server and/or other suitable component/device, both train and execute the trained machine learning model 360. In at least some aspects, a first server and/or other suitable component/device trains the trained machine learning model 360, and a second server and/or other suitable component/device executes the trained machine learning model 360.

### EXAMPLE HUMAN BODY MAP MESH MODEL DISPLAYS

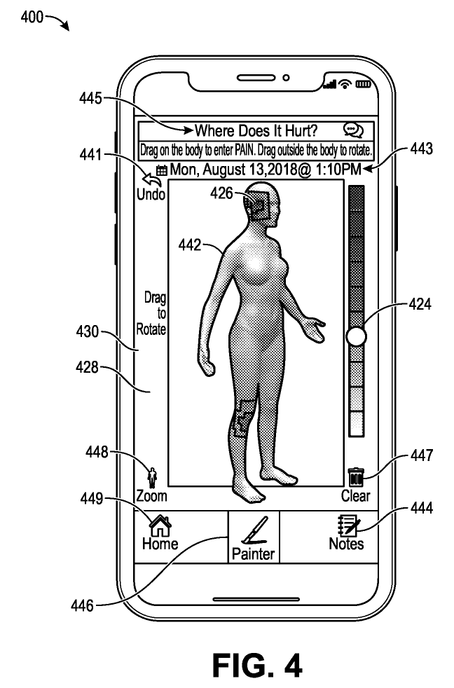

FIG. 4 depicts another example client computing device 400 (“patient device,” e.g., patient device 140 of FIG. 1 and/or patient device 200 of FIG. 2) having a display screen 428 displaying a GUI 430 accessible by a patient (or by user of the patient device 400). The GUI 402 displays a 3-D human body map mesh model 442 indicating real-time information regarding the patient’s condition, e.g., as identified via techniques herein (including, for example, the interactions described with respect to FIG. 2). The display screen 428 of the patient device 400 may display icons to assist the patient in interacting with the many features provided by the GUI 430, such as a home screen icon 449, a “painter” icon 446, a notes icon 444, a clear icon 447, a zoom icon 448, and/or an undo icon 441.

In some examples, the GUI 430 may also display to a patient a natural language prompt 445 which may display one or more identified conditions, prompts, and/or guidance for utilizing the GUI 430. The GUI 430 may also display prompts from the chat bot prompting the user through a series of questions regarding the user’s medical condition, symptoms, and/or other questions regarding the patient’s condition. The “painter” icon 446 displayed on the display screen 428 may allow a patient to interact with the 3-D human body map mesh model 442 to track a location of the patient’s medical condition. As depicted in example FIG. 4, a patient may paint over or select any portion of the 3-D human body map mesh model 442 (e.g., a head location 426, knee, hand, foot, stomach, arm, leg, etc.) to produce output indicating identified symptoms of patient’s medical condition in the body part(s) corresponding to the selected portion of the 3-D human body map mesh model 442 (e.g., to access additional information regarding the head location 426 upon navigating to the head location 426).

In examples, the GUI 430 may also display a slider scale element 424 that allows the patient to select an intensity level within a range by dragging a slider bar across the display screen 428. The display screen 428 may also display color differentiation to distinguish intensity and/or type of pain and/or other conditions. In some examples, the display screen 428 may also display a calendar feature 443 which may display in real-time, a date and/or time of the patient’s update(s) to the 3-D or 4-D human body map mesh model 442. Using these tools of the GUI 430, the patient may input or revise indications of symptoms regarding one or more body parts of the user, e.g., by selecting a particular portion of the patient’s body and indicating an intensity of pain using the slider scale element 424, describe the nature of a symptom via the notes icon 444, etc. In some embodiments, the LLM described herein may process natural language input received via the notes icon 444 to correspondingly manipulate the 3-D human body map mesh model 442 (e.g., to zoom in on or rotate the 3-D human body map mesh model 442, and/or to display the described symptom on a particular portion of the 3-D human body map mesh model 442).

In some embodiments, the 3-D human body map mesh model 442 is a 4-D human body map mesh model, where the patient may access and/or manipulate respective representations of the 3-D human body over time, e.g., via navigation between different dates and times via the calendar feature 443. For example, when the patient has used the techniques described herein to provide a description of one or more symptoms at two different points in time, the user may navigate the calendar feature 443 to display respective versions of the 3-D human body map mesh model 442 respectively corresponding to the two different points in time.

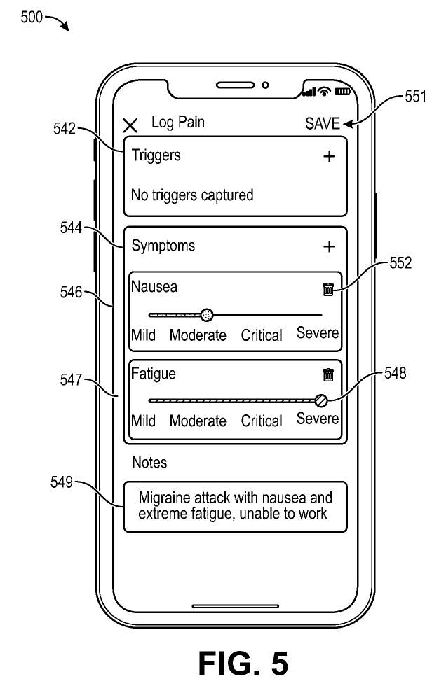

FIG. 5 depicts another example client computing device 500 (“patient device,” e.g., patient device 400 from FIG. 4, and/or another patient device of this disclosure) having a display screen 546 displaying a GUI 547 tracking real-time information regarding the patient’s symptoms associated with the condition, in accordance with some embodiments. A patient may access the GUI 547, for example, via the GUI 430 of FIG. 4 (e.g., via the notes icon 444). In examples, the GUI 547 may also display a “triggers” section 542, via which the patient may identify triggers associated with the patient’s medical condition and/or with one or more symptoms in particular. In further examples, the GUI 547 may also display a “symptoms” section 544, which further displays one or more symptoms describing the patient’s medical condition. The symptoms section 544 may also include an icon for the patient to add further symptoms associated with the patient’s medical condition.

In examples, the GUI 547 may include, one or more slider scale element(s) 548 that allow the patient to select an intensity level in relation to a symptom or condition by dragging a slider bar across the display screen 546. The intensity level displayed via the GUI 547 may display color differentiation to distinguish symptoms, intensity, and/or type of pain and/or other conditions. As shown in example FIG. 5, intensity levels for one or more symptoms may range from mild to severe or numerically (e.g., 0 to 10). Intensity levels may also include moderate and critical.

The GUI 547 may also display a notes section 549 for the patient to input further notes describing the patient’s medical condition, symptoms, etc. In examples, the GUI 547 may also display icons to assist the patient in interacting with the many features provided by the GUI 547, such as a save icon 551 and a trash icon 552. The save icon 551 may allow the patient to save changes after interacting with the GUI 547 to input the patient’s medical condition, triggers, symptoms, notes, etc. as related to their condition.

### EXAMPLE PHYSICIAN COMPUTING DEVICES AND INTERFACES

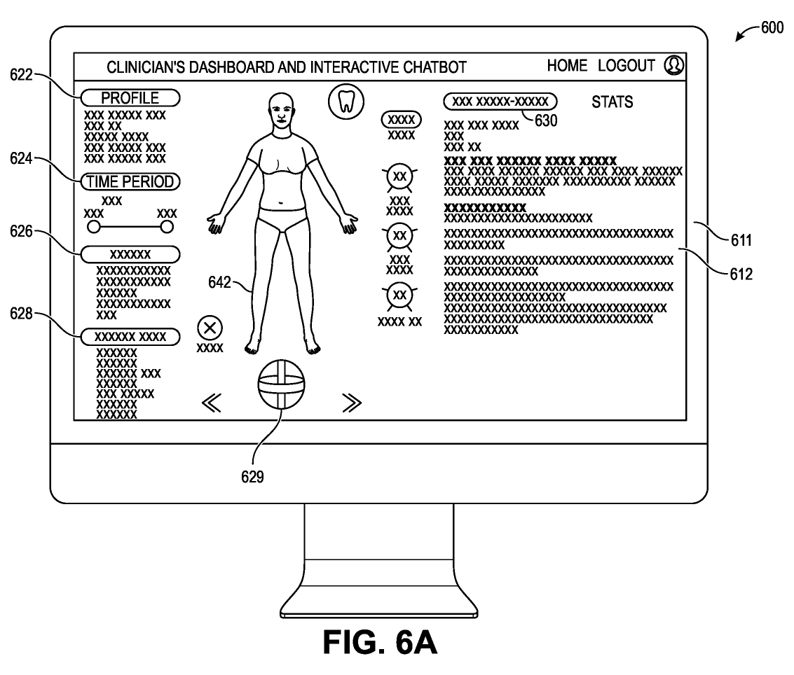

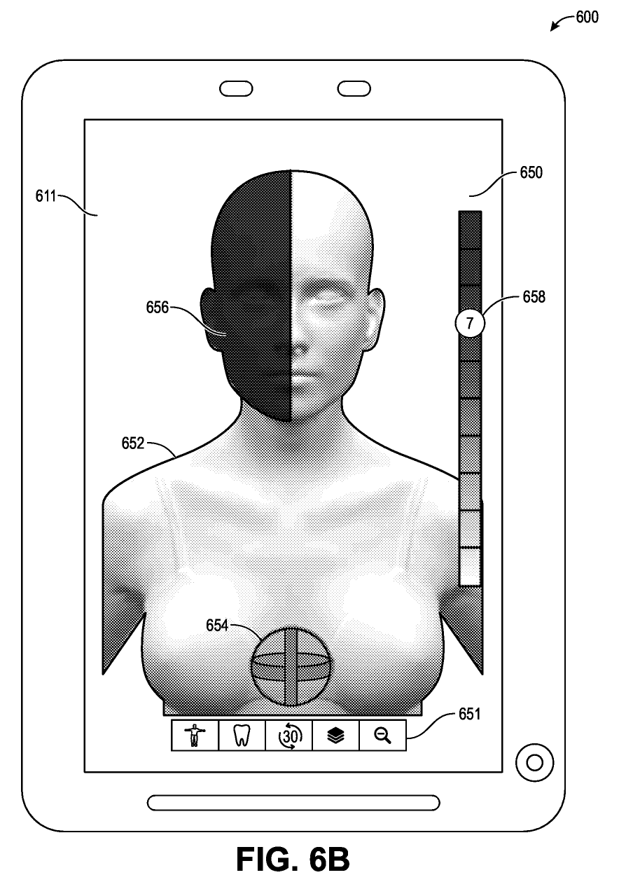

FIG. 6A depicts an example client computing device 600 associated with a physician (“physician device, e.g., physician device 120 of FIG. 1), the physician device 600 displaying patient medical information and/or other information described herein, in accordance with some embodiments. More particularly examples, the physician device 600 may include a display screen 611 executing a GUI 612 which may include a client’s dashboard and enable interactions with a chat bot implementing the LLM described herein. The GUI 612 may also include an icon for a user’s profile, a home button, and/or other icons and buttons to allow easy navigation across the GUI 612. In examples, the GUI 612 may include one or more sections including a profile section 622, a time period section 624, a medication section 626, a book/maintain dates section 628, and/or a chat bot section 630.

The GUI 612 may also display a 3-D or 4-D human body map mesh model 642 which may allow a physician or other user of the physician device 600 to view, track, and/or record aspects of a patient’s medical condition. To facilitate viewing of the 3-D or 4-D human body map mesh model 642, the GUI 612 may include an orbit control element 629 and/or rotation mechanism rotating it around different axes and/or adjust scale of the 3-D or 4-D human body map mesh model 642. The physician may drag the orbit control element 629 across the display screen 611 to manipulate the 3-D or 4-D human body map mesh model 642 depending on one or more areas of focus for the patient’s medical condition. Various other aspects the 3-D or 4-D human body map mesh model 642 may be provided in the GUI 612, including for example any suitable aspects described with respect to FIGS. 4 and/or 5.

In examples, the physician may utilize the chat bot section 630 to provide natural language inputs for example to request medical information regarding the patient’s symptoms or condition, and/or to manipulate the 3-D or 4-D human body map mesh model 442. The physician may provide natural language inputs via a microphone, via text, and/or via other suitable mechanisms described herein. In response, the chat bot may index to the corresponded anatomical region of the 3-D or 4-D human body map mesh model 642 via the display screen 611. As depicted in example FIG. 6A, the chat bot may analyze the physician’s natural language input and, after converting the natural language request for the patient’s condition into clinical data, provide output via the GUI 612.

FIG. 6B depicts another example GUI 650 displayed at the display screen 611 of the physician device 600, in accordance with some embodiments. More particularly, the GUI 650 displays patient medical information including a zoomed in version of a 3-D or 4-D human body map mesh model (which may be the same 3-D or 4-D human body map mesh model 642 from FIG. 6A). In some examples, the GUI 650 includes a navigation bar 651, which may include one or more icons for the physician to easily navigate across the GUI 650. In examples, the icons may include respective icons for viewing, rotating, magnifying, relocating, and/or otherwise manipulating the 3-D or 4-D human body map mesh model 652.

As depicted in example FIG. 6B, the physician may utilize the 3-D or 4-D human body map mesh model 652 for example to log, in real-time, the patient’s medical condition as described by the patient during an in-person office visit. In examples, the physician may also use an orbit control element 654 or rotation mechanism to zoom in to focus on a face portion 656 of the 3-D or 4-D human body map mesh model 652 depicting the patient’s medical condition. In examples, the GUI 650 may include a slider scale element 658 that allows the physician to select an intensity level within a range by dragging a slider bar across the display screen 611. The GUI 650 may utilize color differentiation to distinguish intensity and/or type of pain and/or other conditions of the patient. In the example GUI 650, the 3-D or 4-D human body map mesh model 652 displays a left side of the face portion 656 highlighted with a darker color, e.g. dark red, which may indicate that a patient’s medical condition (e.g., chronic pain) is located on the left portion of the patient’s face and the level of intensity may be indicated as a 7 on a scale ranging from 1 to 10.

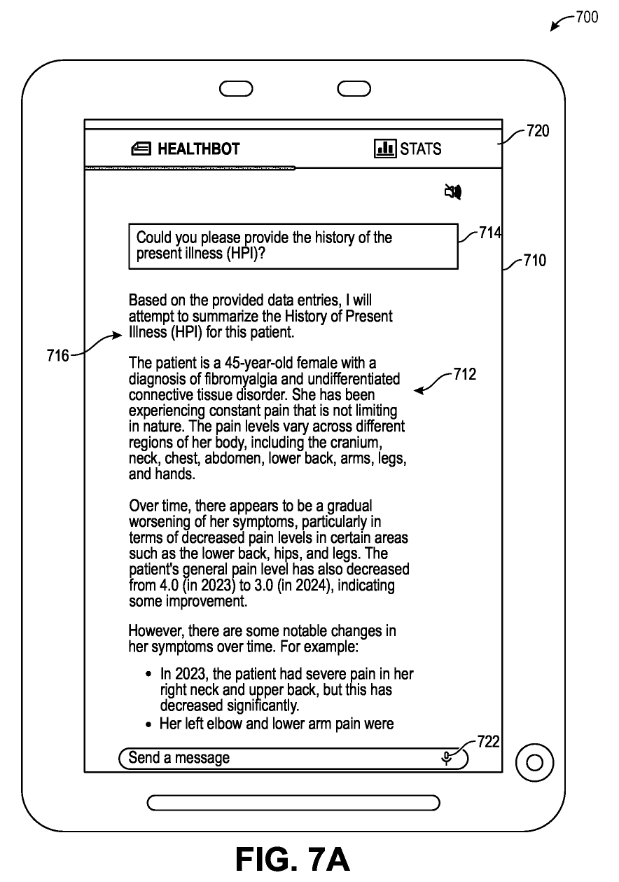

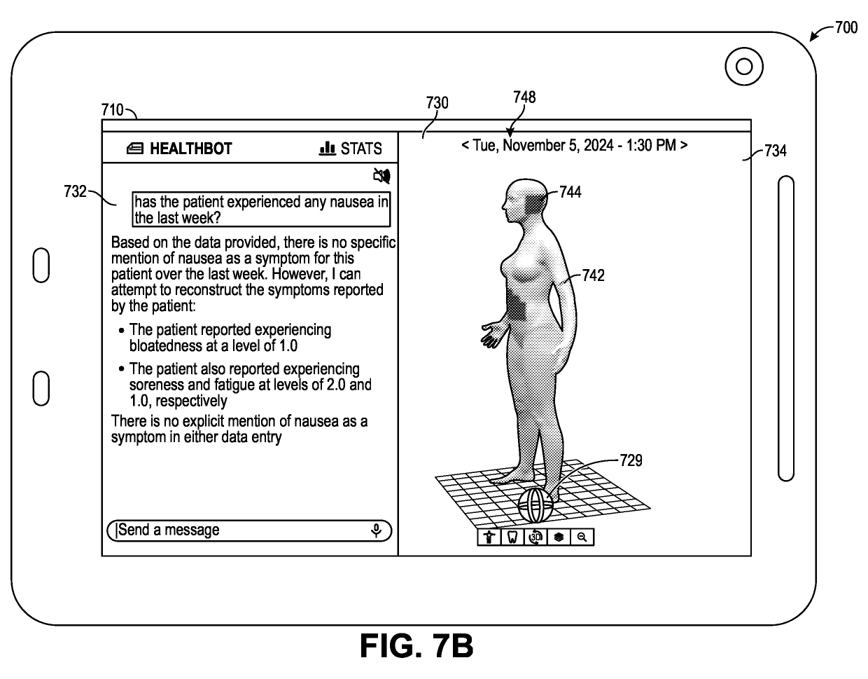

FIG. 7A depicts another example physician device client computing device 700 (e.g., physician device 120 of FIG. 1, and/or physician device 600 of FIGS. 6A and/or 6B) displaying a physician’s live interaction with an active chat bot (“HealthBot”) to retrieve patient medical information associated with a patient, in accordance with some embodiments. The physician may, for example, retrieve the medical information prior to upon beginning an in-person office involving the patient, so as to prepare the physician for the visit by retrieving information associated with the patient.

The physician device 700 includes a display screen 710 executing a GUI 712, via which the physician may interact with an LLM as described herein (e.g., in a similar manner to that described with respect to FIG. 2) to exchange natural language inputs (e.g., a natural language input 714) and outputs (e.g., natural language and/or other outputs, including an output 716). As displayed in the example of FIG. 7A, the physician may for example use the GUI 712 to request, in natural language, information regarding the medical history or condition of a patient. Responsive to the request, the LLM may process the request to identify corresponding structured patient medical information from one or more databases (e.g., patient database 132 from FIG. 1) and generate the output 716 providing patient medical information in a requested or preferred format (e.g., in the form of a structured patient chart, a data table, one or more natural language paragraphs, etc.). In some implementations, the physician may use the GUI 712 to input novel information regarding the patient, including for example information received during an in-office visit, and/or other observations of the physician and/or other medical personnel.

In embodiments, the GUI 712 further includes a navigation bar 720 that may include one or more icons or buttons for navigating between screens, apps, and/or other features displayed on the physician device 700 (e.g., including icons for accessing a chat bot providing LLM functionalities). Analogously to as described with respect to FIG. 2, the physician may use the microphone icon 722 to provide audio input corresponding to the natural language input 714.

FIG. 7B depicts the physician device 700 displaying another example GUI 730, displaying a physician’s live interaction with an active chat bot in combination with a 3-D or 4-D human body map mesh model, in accordance with some embodiments.

More particularly, the GUI 730 includes a chat panel 732, via which the physician may interact with an LLM as described herein (e.g., in a similar manner to that described with respect to the GUI 712 of FIG. 7A). For example, as depicted in FIG. 7B, the physician may request information regarding the presence or absence of a particular condition or symptom in the patient Responsive to the request, the LLM may process the request to identify corresponding structured patient medical information from one or more databases (e.g., patient database 132 from FIG. 1) and generate the output 726 providing patient medical information in a requested or preferred format (e.g., in the form of a structured patient chart, a data table, one or more natural language paragraphs, etc.).

In a model panel 734 of the GUI 730, a 3-D or 4-D human body map mesh model 742 is provided to illustrate medical information of the patient according to the techniques of this disclosure. In embodiments, the model panel 734 manipulates the representation the 3-D or 4-D human body map mesh model 742 based on interactions in the chat panel 732. For example, in response to a natural language input regarding the patient’s head, the model panel 734 may zoom in on a head portion 744 of the 3-D or 4-D human body map mesh model 742. Inversely, in some examples, the chat panel 732 may display output responsive to other manipulations of the 3-D or 4-D human body map mesh model 742 received in the model panel 734. For example, in response to the physician using an orbit control element 729 (and/or other manipulation features such as zoom, rotation, etc.) to focus on the head portion 744, the chat panel 732 may automatically obtain and display output indicating a condition and/or symptom(s) associated with the patient’s head, even absent an explicit request for such region-specific information through the chat panel 732. In embodiments, the physician may utilize a date/time tool 748 to select a particular date and time associated with a patient’s condition, to adapt the 3-D or 4-D human body map mesh model 742 to display the patient’s condition at the selected date/time, and/or to update information in the chat panel 732 accordingly (e.g., to display output reported patient symptoms at the selected date/time).

### OTHER EXAMPLE USER INTERFACES

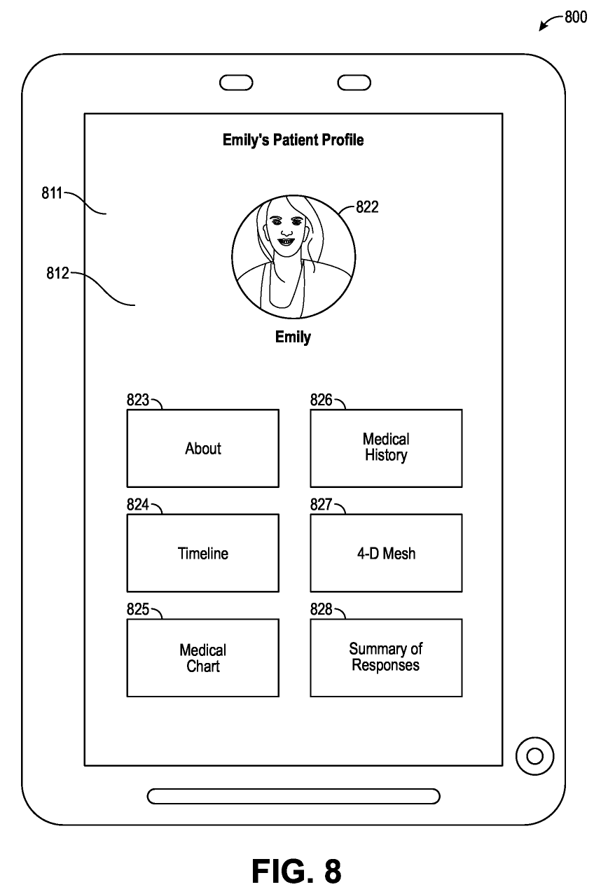

FIG. 8 depicts another example client computing device 800 (e.g., patient device and/or physician device of this disclosure) including a display screen 811 displaying a patient profile GUI 812, in accordance with some embodiments. The patient profile GUI 812 may include a profile photo 822 of the patient, and/or additional page selections such as “about” 823 (which may provide personal information on the patient such as age, date of birth, location, etc.), “timeline” 824 (which may provide specific dates and times of the patient’s medical condition), “medical chart” 825 (which may provide the patient’s medical chart for display), “medical history” 826 (which may provide the patient’s medical history), “4-D Mesh” 827 (which may provide access to a 3-D or 4-D human body map mesh model displaying the patient’s medical condition(s), and/or “summary of responses” 828 (which may provide access to natural language inputs from the patient, e.g., including responses to prompts issued according to techniques of this disclosure).

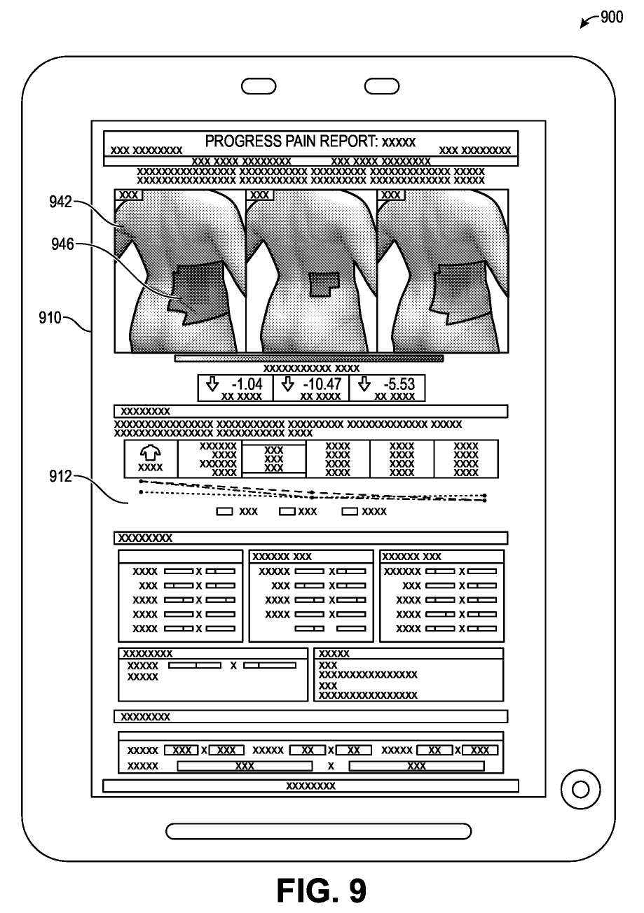

FIG. 9 depicts still another example client computing device 900 (e.g., patient device and/or physician device of this disclosure) having a display screen 910 displaying a GUI 912, in accordance with some embodiments. As depicted in FIG. 9, the GUI 912 displays a 4-D human body map mesh model 942 showing changes in an intensity, type, and/or location of pain in an abdominal region 946 over a duration of time, via simultaneous display of multiple time-based representations of the abdominal region 946.

The GUI 912 may display a pain progress report which may include dates and times of reported symptoms, location/intensity thereof, impacts, treatments, and/or other aspects of the patient’s medical condition (or more particularly, regarding the abdominal region 946). In various embodiments, the GUI 912 may various display charts, graphs, statistics, scales, numbers, averages, etc. as related to the specifics of the patient’s medical condition (e.g., chronic pain).

### EXAMPLE COMPUTER-IMPLEMENTED METHOD

FIG. 10 depicts a flow diagram of another example method 1000, in accordance with some embodiments. The method 1000 may be performed, for example, via one or more servers in communication with a patient device and/or physician device of the present disclosure (e.g., utilizing computing components of FIG. 1 and/or other figures herein). One or more steps of the method 1000 may be implemented as instructions stored on a computer-readable memory (e.g., memory 114, memory 130, memory 152, etc.) and executable on one or more processors (e.g., processor 112, processor 129, processor 150, etc.).

The method 1000 may include, at block 1002, obtaining real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts. In examples, the real-time data regarding the condition of the first user may be obtained by the first user interacting with a chat bot.

Regarding the one or more natural language prompts, in examples, the one or more natural language prompts to the first user may comprise an interactive voice prompt. In other examples, the one or more natural language prompts to the first user may comprise an interactive text prompt. Regarding the one or more natural language responses, in some examples, the one or more natural language responses to the one or more natural language prompts may comprise an interactive voice response. In other examples, the one or more natural language responses to the one or more natural language prompts may comprise an interactive text response.

At block 1004, the method 1000 may also include extracting, using one or more large language models, from the one or more natural language responses, user medical information describing the condition of the first user and conforming to a data structure of a medical record database. For example, the method 1000 may include, wherein the user medical information may include a level of pain and a location of the pain of the user.

At block 1006, the method 1000 may include storing the user medical information in the medical record database.

At block 1008, the method 1000 may also include obtaining, via a second client computing device of a second user, a further one or more natural language responses describing the condition of the first user.

At block 1010, the method 1000 may include, via the one or more large language models, matching the further one or more natural language responses to at least a portion of the user medical information

At block 1012, the method 1000 may also include providing an output indicating the user medical information to the second client computing device of the second user. In some examples, the output may comprise a visual chart of the user’s medical information. In other examples, the output may indicate the user medical information using the one or more natural language responses. In other examples, the output indicating the user medical information may be generated based on the second user interacting with a chat bot.

In some examples, the method 1000 may further include generating a 3-D or 4-D model of the condition of the first user. In examples, the 3-D or 4-D model may be that of a human body map mesh model. In further examples, the human body map mesh model may provide a visual representation of a level of pain and a location of the pain of the first user. In examples, the human body map mesh model may provide a visual representation of a change in the first user’s level of pain and/or location of the pain over a course of time.

Moreover, in some examples, the human body map mesh model may comprise an application, or user interface, for viewing the human body map mesh model, wherein the application, or user interface, may comprise a zooming mechanism for further examination of a particular region on the human body map mesh model. In other examples, the human body map mesh model may comprise an application, or user interface, for viewing the human body map mesh model, wherein the application, or user interface, may comprise a rotation mechanism for further examination of a particular region on the human body map mesh model.

In various embodiments, the method 1000 may include additional, fewer, and/or alternate actions, including various actions described in this disclosure. Moreover, in some embodiments, order of the actions of the method 1000 may vary from that depicted in FIG. 10.

### ADDITIONAL CONSIDERATIONS

While the preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. It is therefore intended that the foregoing detailed description be regarded as illustrative rather than limiting, and that it be understood that it is the following claims, including all equivalents, that are intended to define the spirit and scope of this invention.

By way of example, and not limitation, the present disclosure contemplates at least the following aspects:
1. A computer-implemented method performed via one or more processors, the computer-implemented method comprising: obtaining real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts; extracting, using one or more large language models, from the one or more natural language responses, user medical information describing the condition of the first user and conforming to a data structure of a medical record database; storing the user medical information in the medical record database; obtaining, via a second client computing device of a second user, a further one or more natural language responses describing the condition of the first user; via the one or more large language models, matching the further one or more natural language responses to at least a portion of the user medical information; and providing an output indicating the user medical information to the second client computing device of the second user.
2. The computer-implemented method of aspect 1, wherein the user medical information includes an intensity of pain and a location of the pain of the first user.
3. The computer-implemented method of aspect 1 or aspect 2, further comprising: generating a three dimensional (3-D) or four-dimensional (4-D) model of the condition of the first user.
4. The computer-implemented method of aspect 3, wherein the 3-D or 4-D model is a human body map mesh model.
5. The computer-implemented method of aspect 4, wherein the human body map mesh model provides a visual representation of an intensity of pain and a location of the pain of the first user.
6. The computer-implemented method of aspect 4 or aspect 5, wherein the human body map mesh model provides a visual representation of a change in the intensity of pain and/or location of the pain over a course of time.
7. The computer-implemented method of any one of aspects 4-6, wherein the human body map mesh model comprises a user interface for viewing the human body map mesh model, wherein the user interface comprises a zooming mechanism for further examination of a particular region on the human body map mesh model.
8. The computer-implemented method of any one of aspects 4-7, wherein the human body map mesh model comprises a user interface for viewing the human body map mesh model, wherein the user interface comprises a rotation mechanism for further examination of a particular region on the human body map mesh model.
9. The computer-implemented method of any one of aspects 1-8, wherein the output comprises a visual chart of the user’s medical information.
10. The computer-implemented method of any one of aspects 1-9, wherein the one or more natural language prompts to the first user comprise an interactive voice prompt.
11. The computer-implemented method of any one of aspects 1-10, wherein the one or more natural language prompts to the first user comprise an interactive text prompt.
12. The computer-implemented method of any one of aspects 1-11, wherein the one or more natural language responses to the one or more natural language prompts comprise an interactive voice response.
13. The computer-implemented method of any one of aspects 1-12, wherein the one or more natural language responses to the one or more natural language prompts comprise an interactive text response.
14. The computer-implemented method of any one of aspects 1-13, wherein the output indicates the user medical information using the one or more natural language responses.
15. The computer-implemented method of any one of aspects 1-14, wherein the real-time data regarding the condition of the first user is obtained by the first user interacting with a chat bot.
16. The computer-implemented method of any one of aspects 1-15, wherein the output indicating the user medical information is generated based on the second user interacting with a chat bot.
17. A computer system comprising: one or more processors; and one or more memories storing non-transitory computer-readable instructions that, when executed by the one or more processors, cause the one or more processors to: obtain real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts; extract, using one or more large language models, from the one or more natural language responses, user medical information describing the condition of the first user and conforming the user medical information to a data structure of a medical record database; store the user medical information in the medical record database; obtain, via a second client computing device of a second user, a further one or more natural language responses describing the condition of the first user; via the one or more large language models, match the further one or more natural language responses to at least a portion of the user medical information; and provide an output indicating the user medical information to the second client computing device of the second user.
18. The computer system of aspect 17, wherein the user medical information includes an intensity of pain and a location of the pain of the first user.
19. The computer system of aspect 17 or aspect 18, further comprising: generating a three dimensional (3-D) or four-dimensional (4-D) model of the condition of the first user.
20. The computer system of aspect 19, wherein the 3-D or 4-D model is a human body map mesh model.
21. The computer system of aspect 19 or aspect 20, wherein the human body map mesh model provides a visual representation of an intensity of pain and a location of the pain of the first user.
22. The computer system of aspect 20 or 21, wherein the human body map mesh model provides a visual representation of a change in the intensity of pain and/or location of the pain over a course of time.
23. The computer system of any one of aspects 20-22, wherein the human body map mesh model comprises a user interface for viewing the human body map mesh model, wherein the user interface comprises a zooming mechanism for further examination of a particular region on the human body map mesh model.
24. The computer system of any one of aspects 20-23, wherein the human body map mesh model comprises a user interface for viewing the human body map mesh model, wherein the user interface comprises a rotation mechanism for further examination of a particular region on the human body map mesh model.
25. The computer system of any one of aspects 17-24, wherein the output comprises a visual chart of the user’s medical information.
26. The computer system of any one of aspects 17-25, wherein the one or more natural language prompts to the first user comprise an interactive voice prompt.
27. The computer system of any one of aspects 17-26, wherein the one or more natural language responses to the one or more natural language prompts comprise an interactive voice response.
28. The computer system of any one of aspects 17-27, wherein the one or more natural language responses to the one or more natural language prompts comprise an interactive text response.
29. The computer system of any one of aspects 17-28, wherein the output indicates the user medical information using the one or more natural language responses.
30. The computer system of any one of aspects 17-29, wherein the real-time data regarding the condition of the first user is obtained by the first user interacting with a chat bot.
31. The computer system of any one of aspects 17-30, wherein the output indicating the user medical information is generated based on the second user interacting with a chat bot.
32. A non-transitory computer-readable medium comprising instructions stored thereon, that when executed by a processor, cause the processor to perform steps comprising: obtaining real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts; extracting, using one or more large language models, from the one or more natural language responses, user medical information describing the condition of the first user and conforming to a data structure of a medical record database; storing the user medical information in the medical record database; obtaining, via a second client computing device of a second user, a further one or more natural language responses describing the condition of the first user; via the one or more large language models, matching the further one or more natural language responses to at least a portion of the user medical information; and providing an output indicating the user medical information to the second client computing device of the second user.
33. The non-transitory computer-readable medium of aspect 32, wherein the user medical information includes an intensity of pain and a location of the pain of the first user.
34. The non-transitory computer-readable medium of aspect 32 or aspect 33, further comprising: generating a three dimensional (3-D) or four-dimensional (4-D) model of the condition of the first user.
35. The non-transitory computer-readable medium of aspect 34, wherein the 3-D or 4-D model is a human body map mesh model.
36. The non-transitory computer-readable medium of aspect 34 or aspect 35, wherein the human body map mesh model provides a visual representation of an intensity of pain and a location of the pain of the first user.
37. The non-transitory computer-readable medium of aspect 36, wherein the human body map mesh model provides a visual representation of a change in the intensity of pain and/or location of the pain over a course of time.
38. The non-transitory computer-readable medium of aspect 36 or aspect 37, wherein the human body map mesh model comprises a user interface for viewing the human body map mesh model, wherein the user interface comprises a zooming mechanism for further examination of a particular region on the human body map mesh model.
39. The non-transitory computer-readable medium of any one of aspects 36-38, wherein the human body map mesh model comprises a user interface for viewing the human body map mesh model, wherein the user interface comprises a rotation mechanism for further examination of a particular region on the human body map mesh model.
40. The non-transitory computer-readable medium of any one of aspects 32-39, wherein the output comprises a visual chart of the user’s medical information.
41. The non-transitory computer-readable medium of any one of aspects 32-40, wherein the one or more natural language prompts to the first user comprise an interactive voice prompt.
42. The non-transitory computer-readable medium of any one of aspects 32-41, wherein the one or more natural language responses to the one or more natural language prompts comprises an interactive voice response.
43. The non-transitory computer-readable medium of any one of aspects 32-42, wherein the one or more natural language responses to the one or more natural language prompts comprise an interactive text response.
44. The non-transitory computer-readable medium of any one of aspects 32-43, wherein the output indicates the user medical information using the one or more natural language responses.
45. The non-transitory computer-readable medium of any one of aspects 32-44, wherein the real-time data regarding the condition of the first user is obtained by the first user interacting with a chat bot.
46. The non-transitory computer-readable medium of any one of aspects 32-45, wherein the output indicating the user medical information is generated based on the second user interacting with a chat bot.
47. The method of any one of aspects 1-16, in combination with the method of any other suitable one of aspects 1-16.
48. The system of any one of aspects 17-31, in combination with any other suitable one of aspects 17-31.
49. The non-transitory computer readable medium of any one of aspects 32-46, in combination with any other suitable one of aspects 32-46.
50. Any one of aspects 1-49 in combination with any other suitable one of aspects 1-49.

What is claimed is:

1. A computer-implemented method performed via one or more processors, the computer-implemented method comprising:
obtaining real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts;
extracting, using one or more large language models, from the one or more natural language responses, user medical information describing the condition of the first user and conforming to a data structure of a medical record database;
storing the user medical information in the medical record database;
obtaining, via a second client computing device of a second user, a further one or more natural language responses describing the condition of the first user;
via the one or more large language models, matching the further one or more natural language responses to at least a portion of the user medical information; and
providing an output indicating the user medical information to the second client computing device of the second user.

2. The computer-implemented method of claim 1, wherein the user medical information includes an intensity of pain and a location of the pain of the first user.

3. The computer-implemented method of claim 1, further comprising:
generating a three-dimensional (3-D) or four-dimensional (4-D) model of the condition of the first user, wherein the 3-D or 4-D model is a human body map mesh model that provides a visual representation of an intensity of pain and a location of the pain of the first user.

4. The computer-implemented method of claim 3, wherein the human body map mesh model provides a visual representation of a change in the intensity of pain and/or location of the pain over a course of time.

5. The computer-implemented method of claim 3, wherein the human body map mesh model comprises a user interface for viewing the human body map mesh model, wherein the user interface comprises a zooming mechanism and a rotation mechanism for further examination of a particular region on the human body map mesh model.

6. The computer-implemented method of claim 1, wherein the one or more natural language prompts to the first user comprise an interactive voice prompt or an interactive text prompt.

7. The computer-implemented method of claim 1, wherein the one or more natural language responses to the one or more natural language prompts comprise an interactive voice response or an interactive text response.

8. The computer-implemented method of claim 1, wherein the real-time data regarding the condition of the first user is obtained by the first user or the second user interacting with a chat bot.

9. A computer system comprising:
one or more processors; and
one or more memories storing non-transitory computer-readable instructions that, when executed by the one or more processors, cause the one or more processors to:
obtain real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts;
extract, using one or more large language models, from the one or more natural language responses, user medical information describing the condition of the first user and conforming the user medical information to a data structure of a medical record database;
store the user medical information in the medical record database;
obtain, via a second client computing device of a second user, a further one or more natural language responses describing the condition of the first user;
via the one or more large language models, match the further one or more natural language responses to at least a portion of the user medical information; and
provide an output indicating the user medical information to the second client computing device of the second user.

10. The computer system of claim 9, wherein the user medical information includes an intensity of pain and a location of the pain of the first user.

11. The computer system of claim 9, further comprising:
generating a three dimensional (3-D) or four-dimensional (4-D) model of the condition of the first user, wherein the 3-D or 4-D model is a human body map mesh model that provides a visual representation of an intensity of pain and a location of the pain of the first user.

12. The computer system of claim 11, wherein the human body map mesh model provides a visual representation of a change in the intensity of pain and/or location of the pain over a course of time.

13. The computer system of claim 12, wherein the human body map mesh model comprises a user interface for viewing the human body map mesh model, wherein the user interface comprises a zooming mechanism for further examination of a particular region on the human body map mesh model.

14. The computer system of claim 9, wherein the one or more natural language prompts to the first user comprise an interactive voice prompt.

15. The computer system of claim 9, wherein the one or more natural language responses to the one or more natural language prompts comprise an interactive voice response or an interactive text response.

16. A non-transitory computer-readable medium comprising instructions stored thereon, that when executed by a processor, cause the processor to perform steps comprising:
obtaining real-time data regarding a condition of a first user, the real-time data being received from a first client computing device of the first user by (i) providing one or more natural language prompts to the first user via the first client computing device, and (ii) receiving, from the first client computing device of the first user, one or more natural language responses to the one or more natural language prompts;
extracting, using one or more large language models, from the one or more natural language responses, user medical information describing the condition of the first user and conforming to a data structure of a medical record database;
storing the user medical information in the medical record database;
obtaining, via a second client computing device of a second user, a further one or more natural language responses describing the condition of the first user;
via the one or more large language models, matching the further one or more natural language responses to at least a portion of the user medical information; and
providing an output indicating the user medical information to the second client computing device of the second user.

17. The non-transitory computer-readable medium of claim 16, wherein the user medical information includes an intensity of pain and a location of the pain of the first user.

18. The non-transitory computer-readable medium of claim 16, further comprising:
generating a three dimensional (3-D) or four-dimensional (4-D) model of the condition of the first user, wherein the 3-D or 4-D model is a human body map mesh model.

19. The non-transitory computer-readable medium of claim 18, wherein the human body map mesh model provides a visual representation of an intensity of pain and a location of the pain of the first user.

20. The non-transitory computer-readable medium of claim 18, wherein the human body map mesh model provides a visual representation of a change in the intensity of pain and/or location of the pain over a course of time.
